(19) United States
(12) Patent Application Publication
(10) Pub. No.: US 2022/0387520 A1
(43) Pub. Date: Dec. 8, 2022

(54) METHODS OF TREATMENT OF BLADDER CANCER BY USING MODIFIED BACILLUS CALMETTE-GUÉRIN

(71) Applicants: Texas Biomedical Research Institute, San Antonio, TX (US); The Board Of Regents of The University of Texas System, Austin, TX (US)

(72) Inventors: Russell AULT, San Antonio, TX (US); Niannian JI, Austin, TX (US); Juan I. MOLIVA, San Antonio, TX (US); Robert SVATEK, Austin, TX (US); Jordi B. TORRELLES, San Antonio, TX (US)

(21) Appl. No.: 17/755,008

(22) PCT Filed: Oct. 19, 2020
(86) PCT No.: PCT/US2020/056381

§ 371 Date: Apr. 19, 2022

Related U.S. Application Data
(60) Provisional application No. 62/923,496, filed on Oct. 19, 2019.

(51) Int. Cl. A61K 35/74; A61K 39/39; A61P 35/00

(52) U.S. Cl. CPC A61K 35/74; A61K 39/39; A61P 35/00; A61K 2039/55588

(57) ABSTRACT

Provided are methods for treatment of bladder cancer using compositions of modified Mycobacterium bovis Bacillus Calmette et Guerin (BCG). Also, provided are methods of treatment of non-muscle invasive bladder cancer by intra-bladder injection of these modified BCG compositions. Also, provided are methods of treatment of muscle-invasive bladder cancer by intra-tumor injection of these modified BCG compositions.

### CROSS-REFERENCE TO RELATED APPLICATIONS

This application claims the benefit of, and the priority to, U.S. Provisional Application No. 62/923,496, filed Oct. 19, 2019, which is incorporated by reference herein in its entirety.

### TECHNICAL FIELD

This disclosure relates to methods and compositions of treatment of bladder cancer using modified Bacillus Calmette-Guérin, specifically by modulation of the cell envelope lipids.

### BACKGROUND

Bladder cancer is the most common malignancy of the urinary tract. Around 2.3% of the world population (170 million people) will be diagnosed with bladder cancer at some point during their lifetime, making this disease a major global public health issue. The 2019 American Cancer Society's estimates for bladder cancer in the USA are about 80,470 new cases (61,700 in men/18,770 in women) and 17,670 deaths (12,870 in men/4,800 in women). Bladder cancer is predominantly a disease of the elderly, and with the increase in the aged population, its prevalence is expected to continue to exponentially increase. About 75-85% of patients with bladder cancer present with a disease that is confined to the mucosa or submucosa and thus categorized as non-muscle invasive bladder cancer (NMIBC). In the other 25% of cases, the cancer invades the muscle layer (invasive) and has high rates of metastasis to other sites, often resulting in poor outcomes including death.

The Bacillus of Calmette and Guerin (BCG) strain of Mycobacterium bovis is a live attenuated bacterial vaccine which is used for immunization against Tuberculosis (TB) in endemic areas. Evidence of BCG's efficacy against NMIBC has been established since 1976. TICE® BCG and TheraCys® BCG were approved for treatment of patients with high-grade papillary NMIBC in 1990. About 40-50% of NMIBC patients are deemed high-risk and recommended to receive intravesical BCG. Intravesical BCG is considered the gold standard adjuvant therapy for NMIBC. For early stages of bladder cancer (Stages T0-T1), the current best therapy is surgical removal followed by several treatments with BCG. BCG therapy is ineffective in approximately 30-40% of the cases. Recurrence or relapse after BCG treatment occur in 50% of BCG-treated patients within the first year, 90% relapse within five years. For later stages of bladder cancer (Stages T2-T3), treatment involves complete removal of the bladder. About 5-10% of patients treated with BCG experience significant adverse effects which often lead to discontinuation of treatment. Radical cystectomy is recommended after BCG failure.

### SUMMARY

Disclosed herein are compositions and methods addressing the shortcomings of the art, and may provide any number of additional or alternative advantages, including more effective and less toxic therapy for bladder cancer.

Provided here are modified BCG compositions that contain reduced amounts of cell envelope lipids and methods of treatment of bladder cancer in a subject by administering a therapeutically effective amount of a modified BCG composition. In an embodiment, the modified BCG composition is prepared by exposing BCG bacteria to a delipidating agent and reducing the amount of one or more of trehalose dimycolate (TDM), phenolic glycolipid (PGL), Mycoside B (MycB), tri-acylglycerol (TAG), and phthiocerol dimycocerosates (PDIMs), among others, on the cell envelope of the BCG bacteria. The delipidating agent can be one or more of petroleum ether, sevoflurane, chloroform, methanol, β-d-octyl glucoside, toluene, hexanes, isopropanol, n-butanol, and aqueous acetic acid. In an embodiment, the delipidating agent contains petroleum ether. In an embodiment, greater than 70% of one or more of the TDM, PGL, or MycB has been removed from the cell wall or cell surface of the BCG bacteria by the petroleum ether treatment relative to an untreated control BCG bacteria. In an embodiment, at least 25% of one or more of the TAG and PDIMs have been removed from the cell envelope of the BCG bacteria by the petroleum ether treatment relative to an untreated control BCG composition. In an embodiment, modified delipidated BCG (dBCG) is directly injected into the bladder for treatment of non-muscle invasive bladder cancer. In an embodiment, modified delipidated BCG (dBCG) is directly injected into the bladder for treatment of invasive muscle bladder cancer. In an aspect, the modified BCG composition is administered by intra-tumor injection.

Numerous other aspects, features and benefits of the present disclosure may be made apparent from the following detailed description taken together with the drawing figures. The pharmaceutical compositions can include compounds described herein along with other components, or ingredients depending on desired prevention and treatment goals. It should be further understood that both the foregoing general description and the following detailed description are exemplary and explanatory and are intended to provide further explanation of the invention as claimed.

### BRIEF DESCRIPTION OF THE DRAWINGS

The present disclosure can be better understood by referring to the following figures. The accompanying drawings, which are incorporated in and constitute a part of this specification, illustrate several embodiments and together with the description illustrate the principles of the disclosure.

FIG. 1 is a graphical representation of the bladder weight in mice challenged with MB49 bladder cancer cells via direct bladder wall injection and then treated via direct intratumor injection with modified dBCG composition (marked as “delipidated”) and with phosphate-buffered saline as a negative control.

FIG. 2 is a graphical representation of the bladder weight in mice challenged with MB49 bladder cancer cells via instillation and then treated with conventional BCG (marked as “Std-BCG.TICE”) and modified dBCG (marked as “delipidated”) via instillation, according to an embodiment.

FIG. 3A and FIG. 3B are graphical representations of the increase in amounts of CD45⁺ tumor infiltrating lymphocytes in mice treated with modified dBCG vs. conventional BCG, when evaluated as absolute number (AN) per bladder and AN per 0.1 gram of tissue, respectively.

FIG. 3C and FIG. 3D are graphical representations of the increase in amounts of CD8⁺ tumor infiltrating lymphocytes in mice treated with modified dBCG vs. conventional BCG, when evaluated as AN per bladder and AN per 0.1 gram of tissue, respectively.

FIG. 3E and FIG. 3F are graphical representations of the increase in amounts of bladder cancer specific CD8⁺ tumor infiltrating lymphocytes in mice treated with modified dBCG vs. conventional BCG, when evaluated as AN per bladder and AN per 0.1 gram of tissue, respectively.

FIG. 4 is a graphical representation of the tumor volume in mice challenged with MB49 bladder cancer cells subcutaneously and then treated with intratumor injections of modified dBCG (marked as “BCG. TICE-delipidated”), conventional BCG (marked as “BCG. TICE-original”), and phosphate-buffered saline as a negative control.

### DETAILED DESCRIPTION

Reference will now be made to the embodiments illustrated in the drawings, and specific language will be used here to describe the same. It will nevertheless be understood that no limitation of the scope of the invention is thereby intended. It is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

BCG is the first-line treatment for NMIBC due to its immunomodulatory properties. Tumor removal is performed by scraping of the cancer from the bladder wall via an instrument inserted through the urethra. BCG is then instilled into the bladder via a transurethral catheter (held in the bladder for ˜2 hours). This is performed weekly for 6 weeks initially and then maintenance treatments (weekly×3 weeks) are given every 6 months to help reduce relapses. BCG is thought to act by entering into the damaged bladder wall and stimulating an immune response against remaining cancer cells. This BCG therapy is ineffective in approximately 30-40% of cases and disease recurs in up to 50% of patients. Certain patients with NMIBC cannot complete the multiple required BCG treatments due to developing side effects. Moreover, BCG can sometimes cause life-threatening systemic infection in treated individuals. For invasive bladder cancer, treatment involves complete removal of the bladder. This entails a high risk of complications and results in a significant decrease in quality of life. There are currently no curative treatments for invasive bladder cancer that do not involve removal of the bladder.

The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous. As used herein, unless otherwise noted, the terms “treating”, “treatment” and the like, shall include the management and care of a subject or patient (preferably mammal, more preferably human) for the purpose of combating a disease, condition, or disorder and includes the administration of modified dBCG to prevent the onset of the symptoms or complications, alleviate the symptoms or complications, arrest the development of clinical symptoms, cause regression of clinical symptoms, or eliminate the disease, condition, or disorder.

Disclosed here are compositions of modified dBCG for treatment of bladder cancer. The modified dBCG as described here make it safer for treating bladder cancer. Embodiments of the disclosure also reduce the possibility of life-threatening systemic infections and allow people who develop significant side effects to conventional BCG to undergo further treatments. Otherwise, these people who develop the side effects would not be able to receive additional BCG treatment. For non-invasive bladder cancer, the current best therapy is surgical removal of the tumor followed by several treatments with BCG. Tumor removal is usually performed by scraping of the cancer from the bladder wall via an instrument inserted through the urethra. BCG is then flushed into the bladder, with several repeat BCG treatments. This BCG therapy is ineffective in approximately 30-40% of cases and disease recurs in up to 50% of patients. Improved BCG compositions with better safety profiles can lead to longer progression free survival for this population and delay radical cystectomy.

Disclosed here are methods of treatment of bladder cancer with modified dBCG. The modified dBCG composition is prepared by treatment of the BCG cell envelope to reduce the amount of one or more of the inflammatory and immunomodulatory lipids, such as trehalose dimycolate (TDM), di- and tri-acylglycerols (DAGs/TAGs), phthiocerol dimycocerosates (PDIMs), and phenolic glycolipids (PGLs), among others. These lipids induce rapid and robust innate immune responses that lead to tissue inflammation and damage and/or are used by mycobacteria to subvert host immunity. In an embodiment, the modified dBCG compositions are made by treating BCG with the organic solvent petroleum ether (PE) that selectively extracts many of these inflammatory lipids, including TDM, PGL, mycoside B (MycB), and some triacylglycerols (TAGs) and PDIMs without affecting the viability of BCG. The PE treatment extracts non-polar lipids from BCG cell wall (including its surface) without affecting bacillus viability, attenuating innate inflammatory responses, while allowing for improved adaptive memory responses.

Selective removal of lipids from cell envelopes of current BCG bacteria (generating modified BCG) can reduce severe adverse reactions, which allows the modified BCG treatment to benefit additional 1,200-3,400 patients per year (in USA alone) who are currently intolerant of the therapy. Secondly, use of BCG can be improved and expanded by changing the method of administration. BCG is currently delivered intravesically. Direct delivery (with ultra-sound guidance) to tumor site or bladder wall can improve therapeutic response and expand the use of modified BCG for management of MIBC and delay cystectomy. BCG is currently not part of the standard of care for Muscle-Invasive Bladder Cancer (MIBC). The improved modified BCG product profile may help capture previously BCG-intolerant NMIBC patients, and the changed delivery method can make modified BCG therapy appropriate for MIBC patients. The increased safety of the modified BCG compositions disclosed here render it amenable to new treatment approaches for NMIBC and muscle-invasive bladder cancer. Muscle-invasive bladder cancer is currently treated with total bladder removal, which seriously hinders quality of life. Methods of treatment disclosed here include intratumor injection of modified BCG into muscle-invasive bladder cancer or intrabladder injection for NMIBC. An embodiment of a method of treatment includes an injection of the modified BCG directly into the tumor or the muscle of the bladder. Embodiments include more efficacious and safe treatment of NMIBC by administration of modified BCG. Embodiments include treatment of muscle-invasive bladder cancer that eliminate the need to remove the bladder for muscle-invasive disease by administration of modified BCG. Moreover, administration of modified BCG may be more efficacious for tumors in the bladder itself, using the normal delivery methods. In an embodiment, the modified BCG composition is administered into tumors (subdermal) to dismantle the tumor. Suitable routes of administration include parenteral delivery, including intramuscular, subcutaneous, intra-tumor injections, as well as intrathecal, intravenous, or intraperitoneal injections.

The mechanism of action of BCG in treatment of bladder cancer is undetermined. However, it's believed to promote a local inflammatory reaction with histiocytic and leukocytic infiltration in the urinary bladder that leads to reduction or elimination of superficial cancerous lesions. These inflammatory reactions can cause intolerable immune responses and result in life-threatening systemic infection. To reduce the intolerable side effects, the cell envelopes of BCG, which consists of a complexity of lipids, glycolipids and proteins that interact with macrophages, was selectively modified. In an embodiment, the BCG cell envelope was treated with petroleum ether to reduce the amount of one or more of the inflammatory and immunomodulatory lipids, trehalose dimycolate (TDM), phenolic glycolipid (PGL), Mycoside B (MycB), tri-acylglycerols (TAGs), and phthiocerol dimycocerosates (PDIMs), among others, as these lipids are deemed responsible for inducing immune responses that lead to tissue inflammation and damage. The resulting modified dBCG composition can be injected directly to the cancer site in the bladder, which enable better efficacy. The injection of modified dBCG led to tumor shrinkage in murine models, which corresponds to increased level of bladder cancer specific antigens, such as CD8⁺ and CD45⁺ tumor infiltrating lymphocytes (TILs).

A new mouse model of severe, invasive bladder cancer was also tested. Bladder cancer cells were directly injected into bladder walls of mice, which subsequently developed severe and invasive disease. Then, the modified dBCG composition was administered to the mice with muscle invasive bladder cancer intratumorally with ultrasound guidance. This mouse model was useful for evaluating the efficacy and safety of the modified BCG compositions and determine immune correlates of efficacy.

Embodiments of the modified BCG include BCG compositions, in which the amount of one or more of TDM, PGL, MycB, TAGs, and/or PDIMs on the cell envelope of the BCG has been reduced. The amount of delipidation can vary amongst lipids on the surface of the modified BCG. In an embodiment, the modified BCG (for example, the dBCG) can comprise a biochemical reduction in one or more of TDM, PGL, MycB, TAGs, and/or PDIMs, among others, on the cell envelope. The reduction of one or more of TDM, PGL, MycB, TAG, and/or PDIMs, among others, can independently be at least a 25, 26, 27, 28, 29, 30, 35, 40, 45, 50, 55, 60, 65, 70, 75, 80, 85, 90, 95, 96, 97, 98, 99, or 100 percent reduction. For example, disclosed herein are modified BCG, wherein greater than 60, 65, 70, 75, 80, 85, 90, 95, 96, 97, 98, 99 of the TDM, PGL, and/or MycB have been removed from the cell envelope of the BCG by the petroleum ether treatment relative to an untreated control. In one aspect, the reduction of TDM, PGL, and/or MycB on the cell envelope can be between 60 and 95%, more preferably between 70 and 90%. Also disclosed modified BCG compositions (for example, the dBCG) wherein at least 25, 30, 35, 40, 45, 50, 55, 60, 65, 70% of the TAGs and/or PDIMs has been removed from the cell envelope of the BCG by the petroleum ether treatment relative to an untreated control. In one aspect, the reduction of TAGs and/or PDIMs on the cell envelope can be between 20 and 65%, more preferably between 25 and 50%.

The term “therapeutically effective amount” as used herein, means that amount of the modified BCG composition that elicits the biological response in a tissue system, animal or human that is being sought to treat the disease or disorder. Illustratively, an effective amount of the compositions ranges from nanogram/Kilogram to milligram/Kg amounts for young children and adults. Equivalent dosages for lighter or heavier body weights can readily be determined. The dose should be adjusted to suit the individual to whom the composition is administered and will vary with age, weight and immunological profile of the individual. The exact amount of the composition required will vary from subject to subject, depending on the species, age, weight and general condition of the subject, the particular modified BCG used, its mode of administration and the like. An appropriate amount can be determined by one of ordinary skill in the art using only routine experimentation given the teachings herein. Optimal dosages to be administered may be readily determined by those skilled in the art, and will vary with the particular compound used, the mode of administration, the strength of the preparation, the mode of administration, the number of consecutive administrations within a limited period of time (e.g. every week or every month) and the advancement of the disease condition. In addition, factors associated with the particular patient being treated, including patient age, weight, diet and time of administration, will result in the need to adjust dosages. The term “subject” as used herein, refers to an animal, preferably a mammal, most preferably a human, who has been the object of treatment, observation or experiment. Preferably, the subject has experienced and/or exhibited at least one symptom of the disease or disorder to be treated or prevented.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein. To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

The inventors have previously described methods of modification of Mycobacterium bovis Bacillus Calmette-Guerin (modified dBCG) that improves the safety and efficacy of BCG for prevention of tuberculosis (TB) disease. Specifically, this modification reduces the toxicity of BCG (tissue inflammation) while improving memory immune responses to Mycobacterium tuberculosis, the causative agent of TB. Methods of preparation of the modified BCG as described in PCT Application No. 2018/036649, titled “Delipidated Mycobacterium bovis Bacille Calmette et Guerin (BCG) and Methods of Use” are incorporated herein in their entirety.

The delipidating agent can be any solvent known to remove lipids, including but not limited, to an organic solvent. In an embodiment, the delipidating agent is an aliphatic hydrocarbon, such as petroleum ether, sevoflurane (a nonflammable fluorinated ether), chloroform, methanol, β-d-octyl glucoside, hexanes, toluene, isopropanol, and n-butanol among others, and combinations or more aliphatic hydrocarbons, such as chloroform:methanol and hexanes:isopropanol, among others. In an embodiment, the modified dBCG composition has a reduction of one or more of TDM, PGL, MycB, TAGs, and/or PDIMs on the cell envelope of the BCG due to exposure to a delipidating agent. In an embodiment, the modified dBCG composition has been prepared by exposure to a delipidating agent selected from the group consisting of petroleum ether, sevoflurane (a nonflammable fluorinated ether), chloroform, methanol, β-d-octyl glucoside, hexanes, toluene, isopropanol, n-butanol, and aqueous acetic acid, among others.

The modified dBCG composition results in less inflammation compared to conventional BCG both in vitro and in the in vivo lung environment. For example, lipid extraction from PE treated BCG was assessed by thin layer chromatography (TLC). Due to the hydrophobic nature of PE, significant extraction of PGL and MycB was observed, and these virulence factors are associated with the mycobacterial cell wall (and its surface). Additionally, some PDIMs and TAGs were partially extractable. PE was unable to remove phospholipids such as phosphatidyl-myo-inositol mannosides (PIMs) from the BCG cell envelope, and PIMs were only extractable after extraction with chloroform:methanol (2:1, v/v) (C:M BCG Extract). Large chain carbohydrates such as mannose-capped lipoarabinomannan (ManLAM) were not directly or indirectly extractable with PE and remained on the cell wall (and its surface) of the PE-treated BCG compositions. PE extracted a significant portion of cytotoxic, non-polar molecules from the BCG cell wall (and its surface) with high reproducibility and without affecting modified dBCG viability. Improved protection and reduced pulmonary inflammation (to modified dBCG and M.tb) associated with modified dBCG vaccination is due to the removal of virulent lipids including TDM, MycB, PGL, TAGs, and PDIMs, among others, from the BCG cell wall (and its surface), which can inhibit the development of effective immune responses and/or exacerbate inflammatory responses. TDM has been associated with the ability to inhibit fusion between phospholipid vesicles such as those required for the fusion of phagosomes with lysosomes. Phagosome-lysosome fusion is required for the killing of intracellular M.tb and for the subsequent presentation of foreign peptides along with MHC class II to T cells. TDM can also inhibit cellular energy metabolism, inhibit crosstalk between innate and adaptive immune cells, and induce apoptosis of lymphocytes. Some M.tb clinical isolates can synthesize the trisaccharide form of PGL, which can inhibit Toll-like receptor 2 (TLR2)-induced NF-κB activation, and thus the production of IL-10, TNF, and IL-6. Like PGL, the accumulation of TAGs on the mycobacterial cell envelope can increase virulence. PDIMs also appear to play a role in the virulence of mycobacteria as suggested by M.tb strains that lack PDIM being less virulent in mouse models, a phenotype attributed to reduced binding of M.tb to the plasma membrane of macrophages and increased phagosomal acidification. Other studies have linked the absence of PDIMs on M.tb to reduced survival of M.tb within macrophages, suggesting PDIMs protect M.tb from early innate host responses. Thus, dBCG has a significant impact on multiple virulence factors that influence both innate and adaptive immune elements.

Modified dBCG bacteria may begin rebuilding its cell envelope soon after it enters an environment that is permissive to growth. However, mice vaccinated with modified dBCG had minimal pathology at 150 days post vaccination in the lung, indicating that in the event modified dBCG can resynthesize the removed lipids, this potential reconstitution does not induce the inflammatory response associated with conventional BCG. Moreover, once modified dBCG is inoculated, innate host cells may rapidly begin to kill the bacteria. This rapid killing, combined with a slow antigen presentation process, may explain how modified dBCG is more efficient at controlling M.tb growth. In this context, interestingly, at day 7, modified dBCG vaccination drove a transient decrease in nearly all innate cells studied. This decrease may be attributed to the absence of inflammatory lipids on the modified dBCG cell envelope, where modified dBCG could induce a transient cell turnover to exit the lung, or for the case of monocytes, it could accelerate their differentiation into lung macrophages (alveolar and/or interstitial) and/or dendritic cells. Conversely, the absence of immunomodulatory/inflammatory lipids in modified dBCG could also allow for the development of a stronger adaptive immune response compared to conventional BCG. This is relevant for use of modified dBCG to treat bladder cancer as it has been shown that prior priming of adaptive immune cells, especially CD4⁺ T cells, improves antitumor responses when BCG is instilled in the bladder. This approach is currently being tested for BCG in a Phase 3 implementation clinical trial, wherein bladder cancer patients are given an intradermal BCG vaccination prior to treatment via BCG instillation into the bladder.

The modified dBCG compositions disclosed here can be administered even without a prior vaccination. Improved adaptive immune responses to modified dBCG when instilled in the bladder can improve its efficacy. Uptake of BCG by bladder and/or cancer cells after bladder instillation is required for BCG efficacy in a dose-dependent fashion. Thus, methods that allow direct injection of modified dBCG into bladder or tumor tissue improve upon the efficacy of BCG to treat bladder cancer.

Certain modified dBCG compositions have been used to treat bladder cancer in a subcutaneous model of bladder cancer in mice (using the bladder cancer cell line MB49). These results are being confirmed in nonhuman primate models. These results demonstrate that modified dBCG is efficacious in controlling tumor growth similar to BCG in the subcutaneous tumor model, and they are very effective compared to untreated controls. The anti-tumor efficacy of BCG is partially dependent on the result of the modification. This also suggests modifications of a greater degree than the current modifications will further reduce the potential of BCG to cause side effects in humans. Either of these approaches to improve efficacy or to improve safety (depending on the clinical context) can be accomplished via biochemical means or by genetic alteration to the BCG bacteria.

The modified dBCG compositions can be delivered using several different approaches for the treatment of NMIBC and muscle-invasive bladder cancer. Muscle-invasive bladder cancer is a more advanced stage of bladder cancer and is treated by cystectomy (bladder removal). Embodiments disclosed here include methods of treatment of muscle-invasive bladder cancer by direct injection of modified dBCG into the tumor. For NMIBC, modified dBCG can be injected directly into the bladder wall after removal of the superficial tumor. Avoiding bladder removal would significantly benefit the quality of life of those who are treated for muscle-invasive bladder cancer. Direct injection into the bladder wall also will improve the amount of modified dBCG delivered to bladder tissue for both types of bladder cancer and thus improve efficacy.

Also described here is a new mouse model for investigating bladder cancer therapies. Previous mouse models of bladder cancer involve either adhesion of bladder cancer cell lines to the bladder surface or causing cancer through a lengthy process with carcinogenic chemicals. In both cases, the low number and size of generated tumors do not mimic the severity of human invasive bladder cancer. This limitation has significantly hampered the development of innovative, more effective approaches to treat real patients with bladder cancer. In the new mouse model described here, bladder cancer cells are injected directly into the bladder wall. This procedure gives rise to very severe disease with numerous large tumors and frequent metastasis akin to human bladder cancer. The modified dBCG was then directly injected into the bladder with ultrasound guidance, which translates to minimally invasive methods of treatment. This mouse model of severe, invasive bladder cancer is useful to fully evaluate the efficacy and safety of the modified dBCG compositions and determine immune correlates of efficacy. In another embodiment, the modified dBCG can be injected into the bladder, when bladder cancer cells have previously been instilled and adhered. This resulted in increased CD8⁺ T cell infiltration that are specific to tumor antigens, compared to conventional BCG.

### EXAMPLES

The following Examples are set forth to aid in the understanding of the embodiments of the invention, and are not intended and should not be construed to limit in any way the embodiments set forth in the claims which follow thereafter.

### Example 1

In a preliminary experiment, bladder cancer cells were injected into the bladder wall of mice and subsequently treated with 2 ultrasound-guided injections of the modified dBCG composition. Mice treated with modified dBCG showed a large reduction in bladder tumor burden as measured by bladder weight. In this novel model of severe bladder cancer, control untreated mice had large tumors that had metastasized to the peritoneum and a dramatic loss in total body weight. These results demonstrate that direct intratumor and/or intrabladder wall injection of modified dBCG may be a safe and more efficacious treatment for muscle invasive and NMIBC bladder cancer.

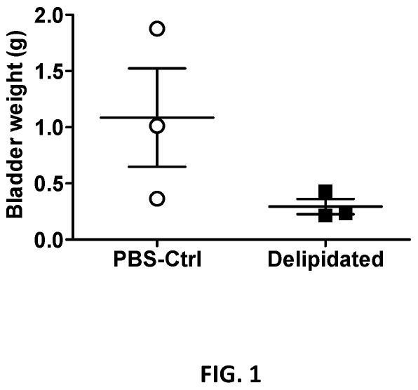

FIG. 1 is a graphical representation of the bladder weight in mice challenged with MB49 bladder cancer cells via direct bladder wall injection and then treated via direct intratumor injection with modified dBCG and with phosphate-buffered saline as a negative control. Mice (n=3 per group) were challenged with MB49 bladder cancer cells via direct bladder wall injection via a surgical process. Mice were then treated twice with either modified dBCG or phosphate-buffered saline via weekly direct intratumor injection under ultrasound guidance (Days 7 and 14 post-challenge). Mice were euthanized at Day 21 post-challenge and total tumor burden quantified by total bladder weight. Mice treated with modified dBCG had dramatically reduced bladder tumor burden compared to mice treated with phosphate-buffered saline.

### Example 2

In a preliminary experiment, bladder cancer cells were instilled into the bladder of mice, and mice were subsequently treated with 3 weekly instillations of modified dBCG or conventional BCG. Mice treated with modified dBCG showed no significant difference in bladder tumor burden, as measured by bladder weight, compared to conventional BCG treatment. However, mice treated with modified dBCG did have more tumor infiltrating lymphocytes, including bladder cancer specific CD8⁺ tumor infiltrating lymphocytes, than mice treated with conventional BCG. This demonstrates that modified dBCG may improve adaptive immune responses to bladder cancer.

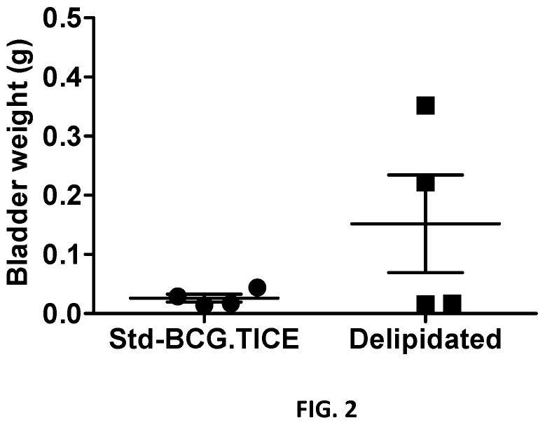

FIG. 2 is a graphical representation of the bladder weight in mice challenged with MB49 bladder cancer cells via instillation and then treated with conventional BCG and modified dBCG via instillation. Mice (n=4 per group) were challenged with MB49 bladder cancer cells via bladder instillation. Mice were then treated with either s conventional BCG or modified dBCG via weekly bladder instillation (Days 1, 8 and 15 post-challenge). Mice were euthanized at Day 19 post-challenge and total tumor burden quantified by total bladder weight. There was not a statistically significant difference in bladder tumor burden between mice treated with conventional BCG vs. modified dBCG.

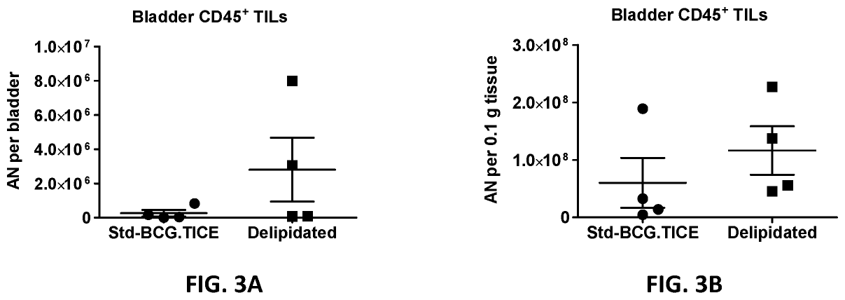

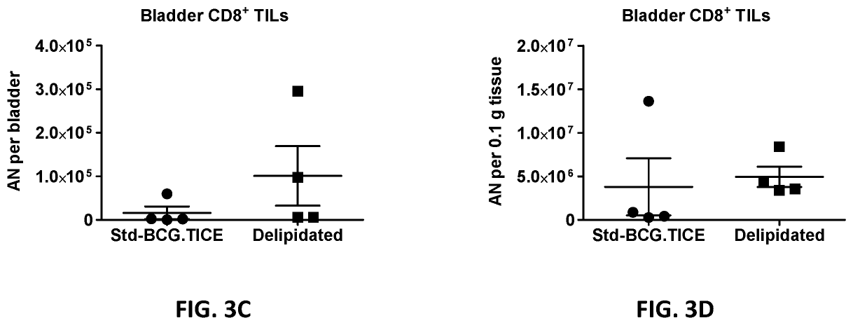

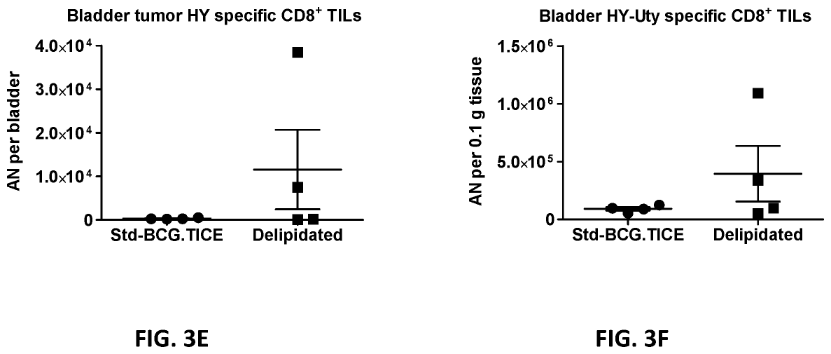

FIG. 3A and FIG. 3B are graphical representations of the increase in amounts of CD45⁺ tumor infiltrating lymphocytes in mice treated with modified dBCG vs. conventional BCG, when evaluated as absolute number (AN) per bladder and AN per 0.1 gram of tissue, respectively. Bladders from the experiment described in FIG. 2 were digested and processed to single cell suspension before staining with antibody-fluorochrome conjugates specific to CD45, CD3 and CD8, a live/dead cell stain and a MHC-I tetramer specific to antigen HY. Data were acquired on a flow cytometer. Tumor infiltrating lymphocytes are quantified as live cells expressing CD45 and CD3. Mice receiving modified dBCG had a trend for increased tumor infiltrating lymphocytes vs. mice receiving conventional BCG.

FIG. 3C and FIG. 3D are graphical representations of the increase in amounts of CD8⁺ tumor infiltrating lymphocytes in mice treated with modified dBCG vs. conventional BCG, when evaluated as AN per bladder and AN per 0.1 gram of tissue, respectively. CD8⁺ tumor infiltrating lymphocytes are quantified as live cells expressing CD45, CD3 and CD8. Mice receiving modified dBCG had a trend for increased CD8⁺ tumor infiltrating lymphocytes vs mice receiving conventional BCG.

FIG. 3E and FIG. 3F are graphical representations of the increase in amounts of bladder cancer specific CD8⁺ tumor infiltrating lymphocytes in mice treated with modified dBCG vs. conventional BCG, when evaluated as AN per bladder and AN per 0.1 gram of tissue, respectively. Bladder cancer specific CD8⁺ tumor infiltrating lymphocytes are quantified as live cells expressing CD45, CD3 and CD8 and staining positive for MHC-I tetramer for HY antigen. Mice receiving modified dBCG had a trend for increased bladder cancer specific CD8⁺ tumor infiltrating lymphocytes vs mice receiving conventional BCG.

### Example 3

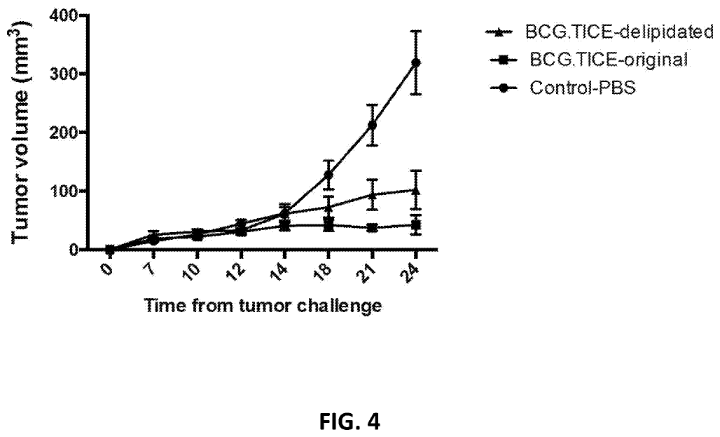

Mice (n=4 per group) were challenged with MB49 bladder cancer cells subcutaneously (2 tumors per mouse) and simultaneously treated by 4 weekly intratumor injections of either modified dBCG, conventional BCG or phosphate-buffered saline. Tumor volume was measured at specified days for 24 days. FIG. 4 is a graphical representation of the tumor volume in mice challenged with MB49 bladder cancer cells subcutaneously and then treated with intratumor injections of conventional BCG, modified dBCG, and phosphate-buffered saline as a negative control. Mice treated with modified dBCG showed a large reduction in tumor volume relative to phosphate-buffered saline treated mice. Mice treated with modified dBCG also showed a large reduction in tumor volume relative to phosphate-buffered saline treated mice, but these mice showed an insignificant trend for increased tumor volume relative to conventional BCG treated mice. Therefore, in this subcutaneous laboratory model of bladder cancer, modified dBCG is efficacious and provides comparable effects to conventional BCG.

Alterations and further modifications of the inventive features illustrated here, and additional applications of the principles of the inventions as illustrated here, which would occur to one skilled in the relevant art and having possession of this disclosure, are to be considered within the scope of the invention. Those skilled in the art will recognize, or be able to ascertain using no more than routine experimentation, many equivalents to the specific embodiments of the invention described herein. Such equivalents are intended to be encompassed by the following claims.

What is claimed is:

1. A method of treatment of bladder cancer in a subject in need thereof, the method comprising: administering a therapeutically effective amount of a Mycobacterium bovis Bacillus Calmette et Guerin (BCG) composition containing reduced amounts of cell envelope lipids.

2. The method of claim 1, wherein the modified BCG composition is prepared by exposing a BCG composition to a delipidating agent and reducing one or more of trehalose dimycolate (TDM), phenolic glycolipid (PGL), Mycoside B (MycB), tri-acylglycerol (TAG), and phthiocerol dimycocerosate (PDIM) as present on cell envelope of the BCG composition.

3. The method of claim 2, wherein the delipidating agent is one or more of petroleum ether, sevoflurane, chloroform, methanol, β-d-octyl glucoside, hexanes, toluene, isopropanol, n-butanol, and aqueous acetic acid.

4. The method of claim 3, wherein the delipidating agent contains petroleum ether.

5. The method of claim 4, wherein presence of one or more of the TDM, PGL, or MycB has been reduced by greater than 70% on the cell envelope of the BCG by the petroleum ether treatment relative to an untreated control.

6. The method of claim 4, wherein presence of one or more of the TAG and PDIM has been reduced by at least 25% of on the cell envelope of the BCG by the petroleum ether treatment relative to an untreated control.

7. The method of claim 1, wherein the bladder cancer is a non-muscle invasive bladder cancer.

8. The method of claim 7, wherein the modified BCG composition is administered by intra-bladder injection.

9. The method of claim 1, wherein the bladder cancer is a muscle invasive bladder cancer.

10. The method of claim 9, wherein the modified BCG composition is administered by intra-tumor injection.
